(19) United States
(12) Patent Application Publication
(10) Pub. No.: US 2024/0424317 A1
(43) Pub. Date: Dec. 26, 2024

(54) METHOD AND DEVICE FOR IMPROVING HEARING ACUITY

(71) Applicant: Soreq Nuclear Research Center, Yavne (IL)

(72) Inventors: Bruno Sfez, Yavne (IL); Ronen Verker, Yavne (IL); Neta Arad-Vosk, Yavne (IL)

(73) Assignee: Soreq Nuclear Research Center, Yavne (IL)

(21) Appl. No.: 18/262,608

(22) PCT Filed: Jan. 25, 2022
(86) PCT No.: PCT/IB2022/050638

§ 371 Date: Jul. 24, 2023

Related U.S. Application Data
(60) Provisional application No. 63/141,563, filed on Jan. 26, 2021.

(51) Int. Cl. A61N 5/06

(52) U.S. Cl. CPC A61N 5/0622; A61N 5/0601; A61N 2005/0605; A61N 2005/063

(57) ABSTRACT

A device for improving auditory acuity of a patient includes an array of waveguides for directing light to the auditory nerve. The array of waveguides is disposed in a flexible structure. A deployment mechanism deploys the structure such that the waveguides are aligned with the auditory nerve. The flexible structure has a shape that is changeable during introduction into the cochlea so as to adapt the shape to a shape of the cochlea.

### FIELD OF THE INVENTION

The present invention relates to a method and device for the improvement of hearing acuity.

### BACKGROUND OF THE INVENTION

It has been shown that optical means can replace electrical means for neural excitation. It has also been demonstrated that this effect can provide much better auditory performance than the electrical counterpart. However the internal structure of the ear is not directly adapted to light guidance and distribution, inter alia, because of the very small angles of curvature of the inner ear. In addition, directing light directly on the nerves requires some kind of alignment that is not easy to perform.

### SUMMARY OF THE INVENTION

The invention comprises several features that aim at solving the main points that have been described in the introduction: the small radius of curvature of the internal ear, and the alignment of the light source and the nerve. This invention enables a large number of excitation channels leading to higher performance and sensitivity to different sound frequencies.

The most straightforward way to guide light into the ear is to use optical fibers. However, optical fibers guide light only if they are bent less than a critical curvature. If the curvature is too high (which can be the case with a small radius of curvature as in the ear), light is no more guided. Typically fibers cannot be bent with a radius of curvature lower than 5 to 10 mm. The cochlea spiral radius of curvature is as low as 2 mm. Therefore light might leave the fiber before arriving at its target. In a first aspect of this invention, light is guided by a metallic waveguide or alternatively a photonic crystal waveguide, as will be described below.

An additional challenge is the alignment of the light waveguide output with the auditory nerve. In the absence of a proper way to align, light is delivered in a wide angular range of directions transverse to the light guide. Only part of the light would impinge on the auditory nerve, and the device energy consumption would be much higher than what is effectively needed. To solve this problem, in a second aspect of the invention, the waveguide is maintained in place using a thermally deformable device, such as a thermally activated shape memory polymer (SMP) device that deforms under heating and maintains the light sources in place facing the auditory nerve, leading to more efficient excitation. Thermally activated SMP can be deformed upon heating from its permanent shape to a temporary shape and maintain this temporary shape upon cooling. Reheating of the polymer results in its returning to its permanent shape.

Finally, it is challenging to introduce the device in a well-controlled manner that is not dependent on the operator manual abilities; it is especially challenging to introduce a straight device in a spiraling tunnel. To solve this problem, in a third aspect of the invention, the device is rolled before use and then unrolled in a controlled manner during the installation.

### BRIEF DESCRIPTION OF THE DRAWINGS

The present invention will be understood and appreciated more fully from the following detailed description, taken in conjunction with the drawings in which:

FIG. 1: 101 is an array of waveguides (three waveguides are represented here for the sake of clarity, but the device can comprise many more waveguides). 102 is light that is coupled into the waveguides. It can be, for example, light coming from an array of lasers. 103 is the output light. Light can be output towards the side of the device, but more conveniently normal to the waveguides plane. This can be obtained by putting a reflective surface that is at 45 degrees from the waveguide facet, as shown in 104, or cutting the waveguide facet at an angle. Light exiting the waveguide can then be focused towards the auditory nerve through a micro-lens that is integrated in the end of the waveguide, as shown in 105.

FIG. 2 shows a possible unfolding of the device once introduced and in order to position it properly. 200 shows the device from above, with the ribs closed. The three elements 2001, 2002 and 2003 represent the waveguides shown in FIG. 1. The closed ribs are above the waveguides set. 201 is a cross section AA of the folded device. 2011 and 2012 illustrate the ribs in the closed position, 2013, 2014 and 2015 illustrate the waveguides cross section. 202 illustrates the deployed device. 203 shows the location of the heating wire and thermistor/thermocouple (dashed line) within the device.

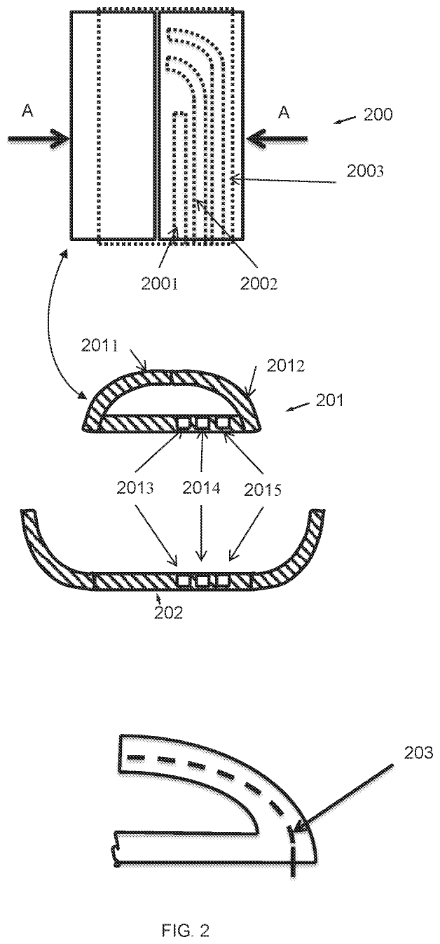

FIG. 3 shows a possible unrolling of the device in order to introduce it within the spiraled-shaped organ. Heating wires and thermistor/thermocouple are located within the central artery of the device (301).

FIG. 4 shows the deployment of the device. At 401, the device is compactly rolled; At 402, the distal part of the device is heated and therefore starts to unroll. By controlling heating at different regions of the device, the device is slowly deployed, in synchronization with the insertion of the device into the organ (403, 404). This control is performed through the online measurement of the wire temperature using the thermistor/thermocouple.

### DETAILED DESCRIPTION OF THE INVENTION

The objective of the device is to bring light to different regions of the auditory nerve through the cochlea. The wavelength of the light used in these embodiments of the invention may be, without limitation, in the near infrared range of approximately 1000 to 1600 nm, or may also include ultraviolet, visible, infrared, far infrared or deep infrared light.

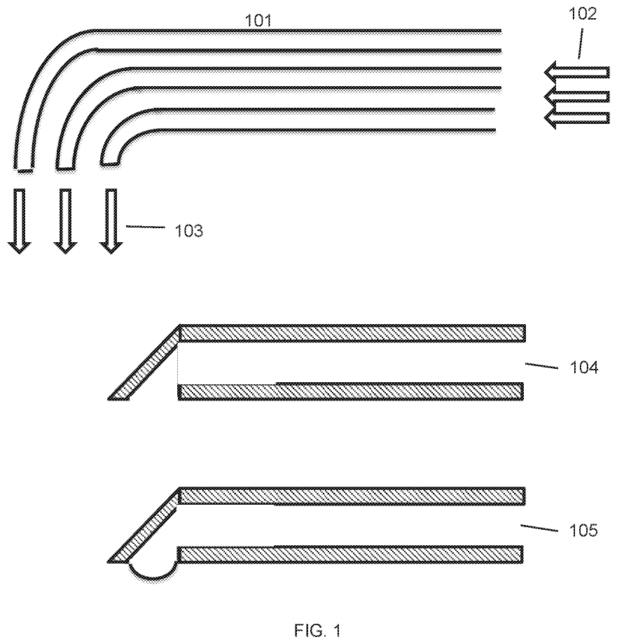

Light is sent to different regions of the auditory nerve using an array of waveguides. One way to do it is, for example, to introduce an array of optical fibers. However, the radius of curvature of the cochlea changes from 8 mm to 2 mm. Such a bending leads light to leave the optical fiber since total internal reflection does not occur. An alternative is to guide light in a total reflecting waveguide structure. In order to do so, it is possible to surround the transparent waveguide by a metallic or by a 1 dimensional photonic crystal (Bragg mirror with high reflective index contrast). Light is therefore guided even when the radius of curvature is very small. In order to deliver light at the right locations, such waveguides of different lengths can be formed to deliver light according to a given location. In FIG. 1 three such waveguides are shown (this is not a limiting number). Light inputs 102 propagate in the metallic waveguides, and the light outputs 103 are delivered at different locations, each waveguide delivering light at a specific location. Light can be delivered in a side direction or redirected in a direction perpendicular to the waveguides plane, towards the auditory nerve, by a deflective mirror 104.

These waveguides are made of a light transparent material (such as optical adhesive from NORLAND or solgel), that possess some elasticity in order to be able to follow the shape of the cochlea. This point will be discussed below.

The transparent waveguide is surrounded either by a metallic surface such as silver, gold or aluminum, by a very low refractive index material (such as fluoride based materials) or by a properly designed alternate of high and low refractive index layers forming a totally reflective one dimensional photonic crystal.

The waveguides are then embedded in a polymer so that the inputs and outputs of the waveguides can be optically accessed. At the output of each waveguide a micro-lens can optionally be positioned so that light that exits the waveguide is focused on the nerve. This micro-lens can be external to the waveguide or alternatively the end facet of the waveguide can be formed so that it has a lens shape (105).

One of the challenges in such a system is to ensure that light is indeed delivered to the auditory nerve. However it is difficult to precisely align the device within the cochlea. In order to assist in this fine alignment, the substrate in which the waveguides are embedded can be an SMP. This SMP is such that below a critical temperature is has the shape 201 and when heated, it takes the shape 202 and stays under this shape when returning below the critical temperature. For example one can take the polymer marketed by SMPTech (Japan) with a critical temperature of 55° C. The device therefore, when heated, deploys ribs like an umbrella and pushes the waveguides against the auditory nerve.

The SMP is heated by embedding heating wires 203 within the polymer. These wires are connected to a low voltage current source that is controlled externally during the installation of the device. When the current is activated, the device deploys its ribs and positions the light source in front of the auditory nerve. The wire temperature is continuously monitored using the thermistor/thermocouple measurement.

The combination of micro-lenses and the stent-like deployment of the device allow using much less energy for nerve activation, which is a critical issue for mobile devices.

Finally, a last issue is the introduction of the device within the cochlea. The device can be identified as a ribbon with optical waveguides embedded within it. The spiral shape of the cochlea is such that it is difficult to precisely guide the ribbon-shaped device within the organ. There are chances that the device will by itself deform and part of the light will arrive opposite to the auditory nerve. Finally, this spiral shape requires the device itself not to be straight but rather take a more complex shape so that it fits the cochlea topology once introduced. This three-dimensional shape can be either a standard or adapted to each patient. The individual cochlea shape can be obtained using standard imaging procedure such as CT or MRI.

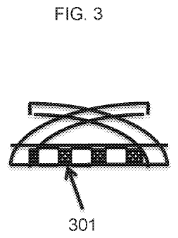

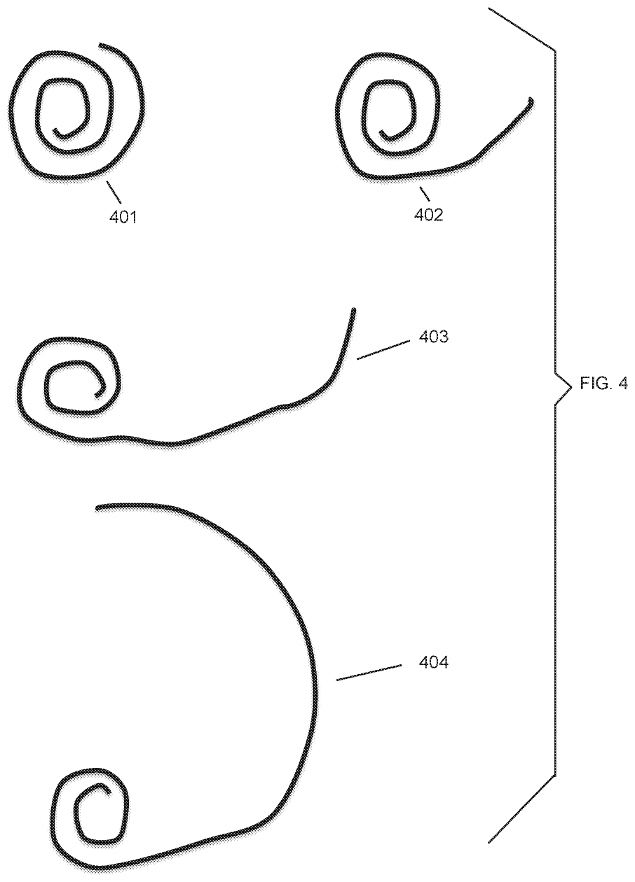

Using the same concept as described above for opening the device, heating wires are positioned along the ribbon as indicated in FIG. 3 (303). The initial shape of the device is for example a rolled shape, and when heat is applied, the ribbon unrolls into a three-dimensional shape that matches the cochlea shape as shown in FIG. 4. FIG. 4 represents the unfolding of the device in two dimensions but in fact the unfolding is three-dimensional, reflecting the complex topology of the cochlea. The ribbon initial shape can similarly be a straight shape that takes the cochlea shape upon heating or whatever shape that is suitable for introduction of the device. As an example, the process is described for a rolled shape.

The heating of the ribbon is not done in one step. Rather, the distal part of the ribbon is first heated and together with the introduction of the device, the ribbon is progressively heated until reaching the proximal region of the device. This can be obtained by introducing heating pads along the ribbon, while transferring the heating current in a low-resistivity wire 303.

In addition, the critical temperature of the ribbon SMP can be chosen to be different from the critical temperature of the ribs (for example, lower), so that unrolling the ribbon does not affect the ribs' opening.

It should be noted that the unrolling of the device and the deployment of the “umbrella” are controlled independently and the heating of one part of the device does not influence the heating of the second part.

Therefore, the procedure is as follows:

First the operator introduces the device by progressively unrolling it by heating the central part of the ribbon (FIGS. 4, 401 to 404).

Once the device is introduced, the operator opens the device by heating the ribs of the device.

1. A device for improving auditory acuity of a patient that has an auditory nerve and a cochlea, the device comprising:
an array of waveguides for directing light to the auditory nerve, said array of waveguides being disposed in a flexible structure;
a deployment mechanism configured to deploy the structure such that the waveguides are aligned with the auditory nerve; and
wherein said flexible structure has a shape that is changeable during introduction into the cochlea so as to adapt the shape to a shape of the cochlea.

2. The device according to claim 1, wherein the waveguides are made of a transparent material surrounded by a reflective surface, and where light is guided by successive reflections on walls of the waveguides.

3. The device according to claim 2, wherein the reflective surface is metallic or a photonic crystal coating.

4. The device according to claim 1, wherein said flexible structure is made of a shape memory material.

5. The device according to claim 1, wherein the shape of said flexible structure is thermally changeable.

6. The device according to claim 1, wherein the device is positioned in place in front of the auditory nerve by unfolding the structure using local heating of the flexible structure.

7. The device according to claim 1, wherein the shape of the flexible structure is modified during the introduction in the ear using local heating of the structure so that it adapts to the cochlear shape.

8. The device according to claim 1, wherein the device permanent shape is individually adapted to each patient by mapping the three-dimensional shape of the cochlea and forming the flexible structure such that after heating it matches the patient cochlea shape and dimensions.
